(19) United States
(12) Patent Application Publication
(10) Pub. No.: US 2022/0298512 A1
(43) Pub. Date: Sep. 22, 2022

(54) SIRNA SEQUENCES TARGETING THE EXPRESSION OF HUMAN GENES JAK1 OR JAK3 FOR A THERAPEUTIC USE

(71) Applicant: COMMISSARIAT A L'ENERGIE ATOMIQUE ET AUX ENERGIES ALTERNATIVES, Paris (FR)

(72) Inventors: Flora CLEMENT, Grenoble (FR); Eric SULPICE, Grenoble (FR); Xavier GIDROL, Grenoble (FR)

(73) Assignee: COMMISSARIAT A L'ENERGIE ATOMIQUE ET AUX ENERGIES ALTERNATIVES, Paris (FR)

(21) Appl. No.: 17/627,606

(22) PCT Filed: Jul. 13, 2020
(86) PCT No.: PCT/EP2020/069768

§ 371 Date: Jan. 14, 2022

(30) Foreign Application Priority Data
Jul. 15, 2019 (FR) 19 07968

(51) Int. Cl. C12N 15/113

(52) U.S. Cl. CPC C12N 15/1137; C12Y 207/10; C12N 2310/14

(57) ABSTRACT

A double-stranded (ds) ribonucleic acid (RNA) comprising a sense strand and an antisense strand, wherein: the sense strand comprises the nucleotide sequence SEQ ID NO: 1 and the antisense strand comprises the nucleotide sequence SEQ ID NO: 2; or the sense stand comprises the nucleotide sequence SEQ ID NO: 3 and the antisense strand comprises the nucleotide sequence SEQ ID NO: 4; or the sense strand comprises the nucleotide sequence SEQ ID NO: 5 and the antisense strand comprises the nucleotide sequence SEQ ID NO: 6; or the sense strand comprises the nucleotide sequence SEQ ID NO: 7 and the antisense strand comprises the nucleotide sequence SEQ ID NO: 8. The dsRNA therefrom may be used as a drug.

### FIELD OF THE INVENTION

The present invention relates to the nucleotide sequences of small interfering RNAs specific to the human genes Janus kinase 1 (JAK1) and Janus kinase 3 (JAK3), respectively. These sequences have been selected so as to have the least possible undesirable or adverse effects while maximising their potential for functional inhibition of the expression of the JAK1 or JAK3 gene, in order to enable therapeutic use thereof in humans.

### TECHNOLOGICAL BACKGROUND

RNA interference was first described in 1998 by Andrew Fire and Craig Mello. This process occurs when a double-stranded RNA has a complementary nucleotide sequence that matches that of an mRNA. The double-stranded RNA thus by virtue of being complementary to the said mRNA then activates the degradation thereof, thereby reducing the expression of the corresponding gene. In detail, the double-stranded RNAs present in a cell are first taken up by a ribonuclease III known as Dicer, the “slicer”. The latter cleaves the double-stranded RNA molecules into fragments of 21 to 25 base pairs. Dicer then transfers the small interfering RNAs (siRNA) to a large multiprotein complex, the RNA-induced silencing complex (RISC). One of the strands of the siRNA, referred to as “passenger”, is eliminated while the other (referred to as “guide”) directs the RISC to the messenger RNAs (mRNAs, or coding RNAs that transmit the message from the gene into the cytoplasm) that have a complementary sequence matching that of the guide strand. If the complementarity (or match) between the siRNA and the target mRNA is perfect, the RISC cleaves the target mRNA which is thus then degraded and is hence no longer translated into protein. A few non-complementary (mismatched) bases are sufficient to prevent cleavage.

The small interfering RNAs, “siRNAs”, are synthetic duplexes of 21 nucleotides that exhibit in particular at positions 2 to 8 of the guide strand in the 5′-3′ direction a “seed” sequence which is particularly important for ensuring specificity thereof. These sequences adhere to certain rules of thermodynamics such as the presence of a guanine or a cytosine in position 1 of the passenger strand, an adenine or a uracil in position 1 of the guide strand, a composition of the “seed” sequence of the guide strand with a minimal guanine/cytosine level, a guanine/cytosine equilibrium for nucleotides 8 to 16 of the guide strand.

In 2018, the siRNA “Patisiran”, marketed by the company Alnylam, was approved for being placed on the market. This siRNA is the first therapeutic siRNA approved for use in humans. The in vitro concentrations conventionally used on cell lines are 10 to 20 nM.

The Janus kinase (JAK) family comprises four non-receptor tyrosine kinases, JAK1, JAK2, JAK3 and Tyk2, which play an essential role in signal transduction induced by cytokines and growth factors. Phosphorylated Janus kinases (JAKs) bind to and activate various signal transducer and activator of transcription (STAT) proteins. These STAT proteins dimerise and then translocate to the nucleus where they act as both signalling molecules and transcription factors and ultimately bind to specific DNA sequences found in promoters of cytokine responsive genes. Various immunodeficiencies and autoimmune diseases such as allergies, asthma, alopecia areata, allograft rejection (allograft), rheumatoid arthritis, amyotrophic lateral sclerosis and multiple sclerosis, as well as solid and hematological cancers result from disruption of signalling in the JAK/STAT pathway.

### SUMMARY OF THE INVENTION

The invention relates to a double-stranded (ds) ribonucleic acid (RNA) comprising a sense strand and an antisense strand wherein:

the sense strand comprises the nucleotide sequence SEQ ID NO: 1 and the antisense strand comprises the nucleotide sequence SEQ ID NO: 2; or

the sense strand comprises the nucleotide sequence SEQ ID NO: 3 and the antisense strand comprises the nucleotide sequence SEQ ID NO: 4; or

the sense strand comprises the nucleotide sequence SEQ ID NO: 5 and the antisense strand comprises the nucleotide sequence SEQ ID NO: 6; or

the sense strand comprises the nucleotide sequence SEQ ID NO: 7 and the antisense strand comprises the nucleotide sequence SEQ ID NO: 8.

The invention also relates to the dsRNA according to the invention for use thereof as a drug.

The invention relates in particular to the dsRNA according to the invention for use thereof in the prevention and/or treatment of a disease associated with a Janus kinase (JAK) gain of function disruption of the JAK signalling pathway.

The invention further relates to a pharmaceutical composition comprising at least one dsRNA according to the invention and a pharmaceutically acceptable carrier.

The invention also relates to an in vitro JAK inhibiting method for inhibiting the expression of Janus kinase 1 (JAK1) or Janus kinase 3 (JAK3) in a cell, the method comprising:

a. introduction into the said human cell of the dsRNA according to the invention; and

b. maintaining of the cell produced in step (a) for a period of time sufficient to achieve degradation of the mRNA of a JAK1 or JAK3 gene, thereby inhibiting the expression of JAK1 or JAK3 in the cell.

### DETAILED DESCRIPTION

The inventors have generated several siRNA sequences that adhere to the main principles of RNA interference. The siRNAs according to the invention have thus been designed in a manner such that:

i. The guide strand of siRNA exhibits 50% to 100% sequence homology with the mRNA of interest (human JAK1 or JAK3);

ii. Known nucleotide motifs for Toll-like receptor (TLR) activation are eliminated;

iii. The 3′ and 5′ GC/AU composition is optimised so as to favour take up of the guide strand in the RISC over the passenger strand;

iv. The potential sequence homologies between the “seed” region of the siRNA guide strand and the three prime untranslated region (3′-UTR) mRNA sequences of the entire genome are reduced as much as possible, in order to minimise the risk of obtaining microRNA-type gene regulatory effects;

v. Preferably, a phosphate is added to the 5′ end of the siRNA guide strand, in order to promote take up of the guide strand by the RISC.

A screening strategy was then developed consisting in comparing in parallel various different siRNA sequences, based on their functional efficacy in inhibiting gene expression, their specificity (no direct modulation of other genes), the reduction in their undesirable or adverse effects (off-target effects, immunogenic effects, potential non-specific effects). The inventors thus selected two siRNA sequences each capable of specifically reducing the expression of the gene and therefore of the human JAK1 or JAK3 protein, without exhibiting undesirable effects (in particular no effect on the expression of JAK2), whereof the common point of interest is the therapeutic potential, for treating a disease associated with a Janus kinase (JAK) gain of function disruption of the JAK signalling pathway, in particular inflammatory diseases (including chronic inflammatory bowel diseases) or certain cancers.

### Definitions

The term “double-stranded RNA” or “dsRNA” denotes a complex of ribonucleic acid molecules having a duplex structure comprising two strands of anti-parallel, substantially complementary nucleic acids. In general, each strand is constituted mainly or entirely of ribonucleotides, but one or both strands may include at least one non-ribonucleotide base, for example a deoxyribonucleotide and/or a modified nucleotide, for example modified by chemical modification. The two strands forming the duplex structure may be different parts of a larger RNA molecule or may be separate RNA molecules (siRNA). When the two strands are different parts of a larger RNA molecule, the 3′ end of one strand may be connected by a nucleotide chain to the 5′ end of the other strand so as to form a hairpin loop structure (short hairpin RNA, or shRNA). Alternatively, the two strands may be connected by a binding means (“linker”) other than a nucleotide chain.

The “antisense strand” refers to the strand of the dsRNA which includes a region of complementarity which is substantially complementary to a target sequence, according to the invention, a target sequence on an mRNA of JAK1 or JAK3.

The “sense strand” refers to the strand of dsRNA that includes a region that is substantially complementary to a region of the antisense strand.

Two sequences whose bases match each other over their entire length are completely complementary. A sequence may also be substantially complementary to a second sequence when the two sequences hybridise and are completely complementary, or are completely complementary over a portion of their length (for example in the case of a double-stranded complex with overhang), or when they have at most 4, 3, 2, or 1 base mismatch.

Throughout the present application, the term “comprising” should be interpreted as encompassing all the characteristic features specifically mentioned, as well as optional, additional, unspecified characteristic features. As used herein, the use of the term “comprising” also describes the embodiment wherein no characteristic features other than the characteristic features specifically mentioned is present (i.e. “consisting of”).

Double Stranded Ribonucleic Acid

As will be described in greater detail below, double-stranded (ds) ribonucleic acid (RNA) molecules are provided in order to inhibit the JAK/STAT signalling pathway, particularly in a human having a disease associated with Janus kinase (JAK) gain of function disruption of the JAK signalling pathway, in which the dsRNA comprises an antisense strand which is complementary to an mRNA resulting from the transcription of a JAK1 or JAK3 gene.

According to one embodiment, the two strands of the dsRNA are separate RNA molecules (siRNA). Each strand of dsRNA comprises 21 nucleotides, and preferably comprises at most 24, 23, or 22 nucleotides. The two strands of the dsRNA are of the same or different lengths, preferably of the same length.

Preferably, each strand of the dsRNA has a 3′ overhang of 2 or more nucleotides, preferably 2 nucleotides.

According to one embodiment, the two strands of the dsRNA are parts of a larger RNA molecule and for example constitute the stem part of a hairpin-like structure. According to this embodiment, the dsRNA preferably comprises at most 60 nucleotides, for example between 55 and 60 nucleotides in total.

According to one embodiment, the dsRNA comprises or consists of a sense strand and an antisense strand, wherein:

the sense strand consists of the nucleotide sequence SEQ ID NO: 1 and the antisense strand consists of the nucleotide sequence SEQ ID NO: 2; or

the sense strand consists of the nucleotide sequence SEQ ID NO: 3 and the antisense strand consists of the nucleotide sequence SEQ ID NO: 4; or

the sense strand consists of the nucleotide sequence SEQ ID NO: 5 and the antisense strand consists of the nucleotide sequence SEQ ID NO: 6; or

the sense strand consists of the nucleotide sequence SEQ ID NO: 7 and the antisense strand consists of the nucleotide sequence SEQ ID NO: 8;

and wherein the nucleotide at the 5′ end of the antisense strand is phosphorylated.

According to one embodiment, the dsRNA is a dsRNA that reduces the expression of the human Janus kinase 3 (JAK3) gene and in which the sense strand comprises the nucleotide sequence SEQ ID NO: 1 and the antisense strand comprises the nucleotide sequence SEQ ID NO: 2 (HJ3D41), or the sense strand comprises the nucleotide sequence SEQ ID NO: 3 and the antisense strand comprises the nucleotide sequence SEQ ID NO: 4 (HMJ3D1). Preferably, in the said dsRNA, the sense strand comprises the nucleotide sequence SEQ ID NO: 1 and the antisense strand comprises the nucleotide sequence SEQ ID NO: 2 (HJ3D41). The dsRNA that includes the sense strand comprising the nucleotide sequence SEQ ID NO: 3 and the antisense strand comprising the nucleotide sequence SEQ ID NO: 4 (HMJ3D1) is in addition specific to the murine JAK3 gene and is capable of reducing the expression of this gene.

According to one embodiment, the dsRNA is a dsRNA that reduces the expression of the human Janus kinase 1 (JAK1) gene and in which the sense strand comprises the nucleotide sequence SEQ ID NO: 5 and the antisense strand comprises the nucleotide sequence SEQ ID NO: 6 (HJ1D2), or the sense strand comprises the nucleotide sequence SEQ ID NO: 7 and the antisense strand comprises the nucleotide sequence SEQ ID NO: 8 (HJ1D8). Preferably, in the said dsRNA, the sense strand comprises the nucleotide sequence SEQ ID NO: 7 and the antisense strand comprises the nucleotide sequence SEQ ID NO: 8 (HJ1D8).

The dsRNA can be chemically modified, in particular so as to increase its stability or to increase the take up of the guide strand by the RISC. Examples of modified dsRNA molecules include dsRNAs containing backbone modifications or internucleotide bonds that are not natural, or containing modified nucleotide bases.

In a preferential manner, the dsRNA is a dsRNA wherein the nucleotide at the 5′ end of the antisense strand is phosphorylated.

The dsRNA according to the invention can also be chemically modified in order to form a conjugate, that is to say covalently bind one or more groups which increase the activity, the cellular distribution or the cellular incorporation of the dsRNA.

The dsRNA may be prepared by any method known to the person skilled in the art, for example by means of chemical synthesis or by means of genetic engineering. In the latter case, the dsRNAs are expressed from transcriptional units inserted into one or more DNA or RNA vectors. These transgenes may be introduced in the form of a linear construct, a circular plasmid, or a viral vector and then incorporated into a host cell where they are integrated into the host cell genome; or else they may be present in the form of an extra chromosomal plasmid. Any viral vector capable of accepting dsRNA encoding sequences may be used, for example vectors derived from adenovirus (AV), adenovirus associated viruses (AA V), retroviruses (eg lentivirus, rhabdovirus), herpes virus, etc. The tropism of the virus can be altered by pseudotyping of the vector, for example, with envelope proteins or other surface antigens derived from other viruses.

The two strands of dsRNA can be transcribed under the control of promoters from two separate expression vectors which are co-transfected into a host cell, or indeed under the control of promoters which are each on the same expression vector. The dsRNA may also be expressed as two inverted repeat sequences joined by a linker polynucleotide sequence to form a stem and loop structure (short hairpin RNA, or shRNA).

Promoters controlling the expression of dsRNA that are present in a DNA plasmid or viral vector may be a eukaryotic RNA polymerase I, II or III promoter (eg U6) or a prokaryotic promoter (eg T7 promoter).

The DNA plasmids for the expression of dsRNA are typically introduced into the target cells by transfection according to the techniques and with the aid of carriers well known to the person skilled in the art. The efficacy of transfection may be monitored, for example, by using a fluorescent label such as green fluorescent protein (GFP) as a reporter gene.

Medical Indications

A dsRNA according to the invention may be used as a drug, in therapy, in particular in human therapy, because of the i-v properties listed above which guided its design and the process by which it was selected by the inventors from among the 43 siRNAs initially generated to block the expression of the human and/or murine JAK1 or JAK3 gene.

The dsRNA is more particularly indicated for the treatment of a disease associated with a Janus kinase (JAK) gain of function disruption of the JAK—particularly JAK1 and/or JAK3—signalling pathway.

A method of treatment of a disease associated with a Janus kinase (JAK) gain of function disruption of the JAK signalling pathway is thus provided, in which a therapeutically effective amount of at least one dsRNA according to the invention is administered to a subject suffering from a disease associated with a Janus kinase (JAK) gain of function disruption of the JAK—particularly JAK1 and/or JAK3—signalling pathway.

The subject is a mammal, preferably a primate, and in a preferential manner a human subject. The subject may also be a rodent, preferably a mouse.

The diseases associated with a Janus kinase (JAK) gain of function disruption of the JAK—particularly JAK1 and/or JAK3—signalling pathway, include in particular immune-mediated inflammatory diseases, including in particular chronic inflammatory diseases of the bowel (Crohn's disease and ulcerative colitis), rheumatoid arthritis, alopecia areata, uveitis, atopic dermatitis, ankylosing spondylitis, psoriasis, lupus erythematosus, lupus nephritis, myelofibrosis, transplant rejection and graft versus host disease; and cancers, notably leukemias (in particular acute lymphoblastic leukemia (ALL), and acute myelogenous leukemia (AML)) and solid tumour cancers. In a preferential manner, a dsRNA according to the invention is indicated for the treatment of immune-mediated inflammatory diseases, including in particular chronic inflammatory diseases of the bowel, myelofibrosis, transplant rejection, and cancers.

For the therapeutic use thereof, the dsRNA is generally formulated in a pharmaceutical composition that comprises at least one dsRNA according to the invention and a pharmaceutically acceptable carrier.

According to one embodiment, the pharmaceutical composition comprises at least one dsRNA that is a JAK 1 inhibitor according to the invention.

According to another embodiment, the pharmaceutical composition comprises at least one dsRNA that is a JAK 3 inhibitor according to the invention.

According to yet another embodiment, the pharmaceutical composition comprises at least one JAK 1-inhibitor dsRNA and at least one JAK 3-inhibitor dsRNA according to the invention, for example:
a dsRNA wherein the sense strand comprises the nucleotide sequence SEQ ID NO: 1 and the antisense strand comprises the nucleotide sequence SEQ ID NO: 2 (HJ3D41), and (i) a dsRNA wherein the sense strand comprises the nucleotide sequence SEQ ID NO: 7 and the antisense strand comprises the nucleotide sequence SEQ ID NO: 8 (HJ1D8) or (ii) a dsRNA wherein the sense strand comprises the nucleotide sequence SEQ ID NO: 5 and the antisense strand comprises the nucleotide sequence SEQ ID NO: 6 (HJ1D2); or
a dsRNA wherein the sense strand comprises the nucleotide sequence SEQ ID NO: 3 and the antisense strand comprises the nucleotide sequence SEQ ID NO: 4 (HMJ3D1), and (i) a dsRNA wherein the sense strand comprises the nucleotide sequence SEQ ID NO: 7 and the antisense strand comprises the nucleotide sequence SEQ ID NO: 8 (HJ1D8) or (ii) a dsRNA wherein the sense strand comprises the nucleotide sequence SEQ ID NO: 5 and the antisense strand comprises the nucleotide sequence SEQ ID NO: 6 (HJ1D2).

The pharmaceutical composition according to the invention may also comprise at least one dsRNA according to the invention, at least one other active ingredient, for example an anticancer agent, and a pharmaceutically acceptable carrier.

According to yet another embodiment, the invention relates to a JAK 1-inhibitor dsRNA according to the invention for use thereof as a drug, or for use thereof in the prevention and/or the treatment of a disease associated with a Janus kinase (JAK) gain of function disruption of the JAK signalling pathway, in combination with a JAK 3-inhibitor dsRNA according to the invention or another active ingredient, for example an anticancer agent. According to this embodiment, the JAK 1-inhibitor dsRNA, and the JAK 3-inhibitor dsRNA according to the invention or the other active ingredient, may be formulated in the same given pharmaceutical composition or else indeed separately, in separate individual pharmaceutical compositions. They may be administered simultaneously or separately, for example in a manner so as to be spaced out over time. According to this embodiment, the JAK 1-inhibitor dsRNA is administered to a subject who is being treated with a JAK 3-inhibitor dsRNA according to the invention or another active ingredient.

According to yet another embodiment, the invention relates to a JAK 3-inhibitor dsRNA according to the invention for use thereof as a drug, or for use thereof in the prevention and/or the treatment of a disease associated with a Janus kinase (JAK) gain of function disruption of the JAK signalling pathway, in combination with a JAK 1-inhibitor dsRNA according to the invention or another active ingredient, for example an anticancer agent. According to this embodiment, the JAK 3-inhibitor dsRNA, and the JAK 1-inhibitor dsRNA according to the invention or the other active ingredient, may be formulated in the same given pharmaceutical composition or else indeed separately, in separate individual pharmaceutical compositions. They may be administered simultaneously or separately, for example in a manner so as to be spaced out over time. According to this embodiment, the JAK 3-inhibitor dsRNA is administered to a subject who is being treated with a JAK 1-inhibitor dsRNA according to the invention or another active ingredient.

The pharmaceutical composition is formulated based on its route of administration which may be, for example, local administration or systemic administration, such as the parenteral route, or the intravenous (iv) route.

The pharmaceutical composition is administered with a sufficient dosage of dsRNA to induce inhibition of the expression of JAK1 and/or JAK3.

Determining the route of administration and the appropriate dosage depending on the subject is well within the capabilities of the person skilled in the art.

Method for Inhibiting JAK1 or JAK3 in a Cell

The application also describes a JAK inhibiting method, either in vivo or in vitro, for inhibiting the expression of Janus kinase 1 (JAK1) or Janus kinase 3 (JAK3) in a cell, the method comprising:

a. introduction into the said human cell of the dsRNA according to the invention; and

b. maintaining of the cell produced in step (a) for a period of time sufficient to achieve degradation of the mRNA of a JAK1 or JAK3 gene, thereby inhibiting the expression of JAK1 or JAK3 in the cell.

The cell is preferably a human or murine cell.

According to one embodiment, in step a, the dsRNA is brought into contact with the said cell at a concentration greater than or equal to 5 pM, preferably from 5 pM to 10 nM, or indeed even from 5 pM to 1 nM, or indeed even from 5 pM to 100 pM, or indeed even from 5 pM to 50 pM, yet more preferably from 5 pM to 25 pM.

A transfection reagent (for example, cationic lipids, such as Lipofectamine) is typically used so as to facilitate transfection of the cell with the dsRNA.

When the method is implemented in vitro, in step b) the cell is maintained in a culture medium suitable for its survival and/or propagation. The selection of such a medium is well within the capabilities of the person skilled in the art.

The present invention will be illustrated in greater detail by means of the figures and examples here below.

### FIGURES

FIG. 1 represents the typical structure of an siRNA according to the invention, comprising a 3′ overhang, of 2 unpaired nucleotides, and preferably a phosphate group at the 5′ end of the guide strand. The passenger strand corresponds to the sense strand, and the guide strand to the antisense strand.

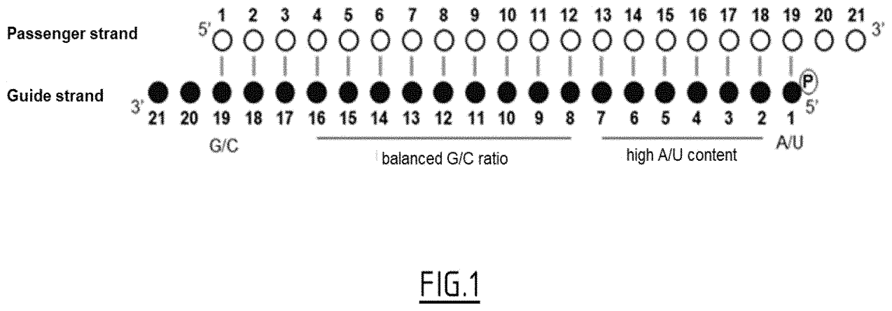

FIG. 2 represents the hybridisation regions, on the JAK1 gene encoding part, of the different JAK1-inhibitor siRNAs tested.

FIG. 3 represents the hybridisation regions, on the JAK3 gene encoding part, of the different JAK3-inhibitor siRNAs tested

FIG. 4 presents the results of 3 independent experiments for measuring the level of JAK1, JAK2, JAK3 or TYK2 gene expression in Caco-2 cells transfected with 10 nM of siRNA (targeting JAK1).

FIG. 5 presents the results of 3 independent experiments for measuring the level of JAK1, JAK2, JAK3 or TYK2 gene expression in Caco-2 cells transfected with 10 nM of siRNA (targeting JAK3).

FIG. 6 presents the results of quantification of JAK1 expression in PC3 cells transfected with 12.5 pM of siRNA.

FIG. 7 presents the results of quantification of JAK3 expression in PC3 cells that stably overexpress JAK3, and were transfected with 1 nM of siRNA.

FIG. 8 shows the analysis by Western blot of JAK1, JAK3, or GAPDH expression in PC3 cells that stably overexpress JAK3, and were transfected with 0.5 pM to 12.5 pM of siRNA.

FIG. 9 shows the expression of potential target genes in T47D cells transfected with 10 nM of siRNA HJ1D2 targeting JAK1 or a control siRNA (siAS).

FIG. 10 shows the expression of potential target genes in T47D cells transfected with 10 nM of siRNA HJ1D8 targeting JAK1 or a control siRNA (siAS).

FIG. 11 shows the expression of potential target genes in T47D or PC3 cells transfected with 10 nM of siRNA HJ3D41 targeting JAK3 or a control siRNA (siAS).

FIG. 12 shows the expression of potential target genes in T47D cells transfected with 10 nM of siRNA HMJ3D1 targeting JAK3 or a control siRNA (siAS).

FIG. 13 shows the effect of the siRNAs according to the invention or control on cell proliferation (a), adenosine triphosphate (ATP) metabolism (b), and apoptosis (c), and (d). “opti” denotes the medium OPTI-MEM used to produce the mixture during transfection, and “lipo” denotes the transfection agent used (LipofectamineRNAimax).

### EXAMPLES

### Example 1: Identity of siRNAs

Several siRNA sequences designed to inhibit expression of the human, or human and murine JAK1 or J1K3 gene, were compared with each other as well as with several commercially available siRNA sequences known to inhibit expression of the human JAK1 or JAK3 genes. The nomenclature of siRNAs is as follows: H for Homo Sapiens (the species concerned), M for Mus musculus, J1 for JAK1: targeted transcript (and J3 for JAK3), then a unique number Dx to characterise the sequence. Thus siHJ1D8 is the siRNA sequence which targets the human JAK1 gene, the unique number whereof is D8. siHMJ3D1 is the siRNA sequence that targets the human and murine JAK3 genes, the unique number whereof is D8. “A”, “Q” and “H” denote the siRNAs marketed respectively by the companies Ambion, Qiagen, and Dharmacon.

Several experimental approaches have been combined with a view to providing two sequences of siRNA that have the greatest efficacy and specificity possible while presenting the least possible undesirable or adverse effects such as to make possible the therapeutic use thereof (see Tables 1-2).

TABLE 1
siRNA Sequences Targeting Human JAK3

| | | Antisense |
|---|---|---|
| | Sense Sequence | Sequence |
| Name | (Passenger) | (Guide) |
| HJ3D41 | CGACUUUCCA | UACGAUUUCU |
| | GAAAUCGUAG | GGAAAGUCGC |
| | A | A |
| | (SEQ ID | (SEQ ID |
| | NO: 1) | NO: 2) |
| HMJ3D1 | GCGUGGAGCU | UAGCGGCACA |
| | GUGCCGCUAU | GCUCCACGCU |
| | G | G |
| | (SEQ ID | (SEQ ID |
| | NO:3) | NO: 4) |

TABLE 2
siRNA Sequences Targeting Human JAK1

| | | Antisense |
|---|---|---|
| | Sense Sequence | Sequence |
| Name | (Passenger) | (Guide) |
| HJ1D2 | GGAUUACAAG | UUCGUCAUCC |
| | GAUGACGAAG | UUGUAAUCCA |
| | G | U |
| | (SEQ ID | (SEQ ID |
| | NO: 5) | NO: 6) |
| HJ1D8 | GGACAUCAGC | UCGCUUGUAG |
| | UACAAGCGAU | CUGAUGUCCU |
| | A | U |
| | (SEQ ID | (SEQ ID |
| | NO: 7) | NO: 8) |

The inventors compared dozens of original or already marketed sequences, in order to be able to derive therefrom two siRNAs capable of targeting JAK1 or JAK3 for therapeutic use.

It is interesting to note that among the siRNAs generated according to the principles of RNA interference, the siRNA sequence siHJ1D64 was in fact not effective in inhibiting the expression of JAK1.

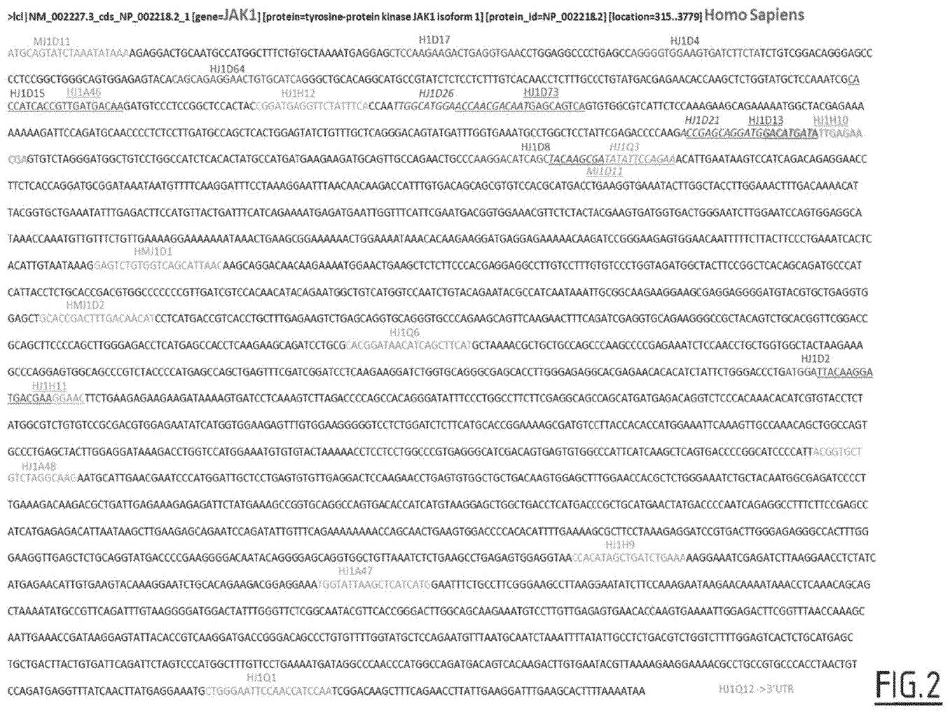

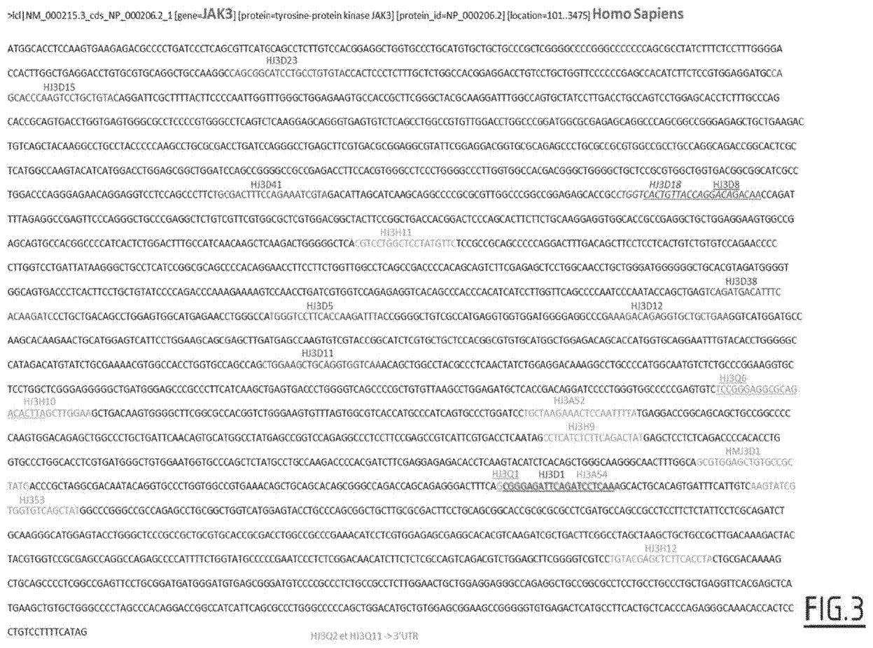

The position of the tested siRNAs on the JAK1 and JAK3 genes is shown in FIGS. 2 and 3.

TABLE 3
Synthesis of the experiments carried out to test the siRNAs directed against human JAK1.

| | | | | | | | | | | | | Average |
|---|---|---|---|---|---|---|---|---|---|---|---|---|
| | | | | | | | | RT-QPCR | | | | Weighted |
| | Western Blot | | | | | | | | | | Average | Efficacy |
| | Caco2 | Caco2 | | Caco2 | Caco2 | Caco2 | Average | Caco2 | Caco2 | Caco2 | Inhibition | Score |
| | with | with | Caco2 | with | with | with | Inhibition | with | with | with | of Jak1 | WB |
| | 10 nM | 10 nM | with | 0.2 nM | 0.2 nM | 0.2 nM | of Jak1 at | 0.2 nM | 0.2 nM | 0.2 nM | mRNA at | 0.2 nM@3, |
| | siRNA | siRNA | 1 nM | siRNA | siRNA | siRNA | 0.2 nM | siRNA | siRNA | siRNA | 0.2 nM | QPCR |
| | Exp1 | Exp2 | siRNA | Exp1 | Exp2 | Exp3 | Exp1-2-3 | Exp1 | Exp2 | Exp3 | n = 3 | 0.2 nM@2 |
| | siRNA | | | | | | | | | | | |
| | | | | | | | | | | | % | |
| | | | | | | | Average | % | | | Average | |
| | | | | | | % | Inhibition | inhib. | % inhib. | % inhib. | inhib. | Weighted |
| | % inhib. | % inhib. | % inhib. | % inhib. | % inhib. | inhib. | of Jak1 at | Jak1 | Jak1 | Jak1 | Jak1 | Efficacy |
| | Jak1 | Jak1 | Jak1 | Jak1 | Jak1 | Jak1 | 0.2 nM | mRNA | mRNA | mRNA | mRNA | Score |
| AS | 0.0 | 0.0 | 0.0 | 0.0 | 0.0 | 0.0 | 0.0 | 0.0 | 0.0 | 0.0 | 0.0 | 0.0 |
| HJ1A46 | 96.7 | NA | 92.3 | 73.8 | NA | NA | NA | 58.8 | NA | NA | NA | NA |
| HJ1A47 | 97.5 | NA | 93.2 | 82.0 | NA | NA | NA | NA | NA | NA | NA | NA |
| HJ1A48 | NA | 97.0 | 91.3 | 33.5 | NA | NA | NA | NA | NA | NA | NA | NA |
| HJ1Q1 | 94.0 | 89.4 | 91.6 | 72.1 | NA | NA | NA | NA | NA | NA | NA | NA |
| HJ1Q3 | 95.1 | 96.1 | 90.2 | 69.1 | NA | NA | NA | NA | NA | NA | NA | NA |
| HJ1Q6 | 94.9 | 93.7 | 93.7 | 78.2 | NA | NA | NA | 70.6 | NA | NA | 70.6 | NA |
| HJ1Q12 | 95.0 | 88.8 | 83.4 | 72.3 | NA | NA | NA | 0.0 | NA | NA | 0.0 | NA |
| HJ1H9 | 96.7 | 98.9 | 85.0 | 78.1 | 82.1 | 88.2 | 82.8 | 70.1 | 45.9 | 89.0 | 68.3 | 77.0 |
| HJ1H10 | 80.2 | 85.6 | NA | NA | NA | NA | NA | NA | NA | NA | NA | NA |
| HJ1H11 | 92.3 | 92.9 | 81.8 | 62.7 | NA | NA | 62.7 | NA | NA | NA | NA | NA |
| HJ1H12 | 94.6 | 90.3 | 83.6 | 75.1 | NA | NA | 75.1 | NA | NA | NA | NA | NA |
| HJ1D2 | NA | NA | 91.2 | 76.5 | 94.2 | 92.7 | 87.8 | 77.8 | 91.4 | 90.6 | 86.6 | 87.3 |
| HJ1D4 | NA | NA | 93.1 | 85.5 | 97.0 | 91.9 | 91.5 | 73.1 | 80.7 | 88.3 | 80.7 | 87.1 |
| HJ1D8 | NA | NA | 79.3 | 88.3 | 93.5 | 94.9 | 92.2 | 71.0 | 85.2 | 93.3 | 83.2 | 88.6 |
| HJ1D13 | NA | NA | 88.0 | 76.6 | 61.7 | 95.9 | 78.1 | 43.5 | 55.8 | 83.9 | 61.0 | 71.3 |
| HJ1D15 | NA | NA | 88.8 | 80.3 | 79.3 | 98.0 | 85.9 | 43.0 | 79.9 | 86.7 | 69.9 | 79.5 |
| HJ1D17 | NA | NA | 87.7 | 74.3 | 96.7 | 99.9 | 90.3 | 48.4 | 84.0 | 83.5 | 71.9 | 83.0 |
| HJ1D21 | NA | NA | 84.0 | 81.2 | 92.7 | 99.5 | 91.1 | 50.2 | 83.3 | 90.3 | 74.6 | 84.5 |
| HJ1D26 | NA | NA | 76.5 | 65.3 | 40.7 | 97.5 | 67.8 | 37.9 | 74.5 | 63.1 | 58.5 | 64.1 |
| HJ1D64 | NA | NA | −7.9 | NA | NA | NA | NA | −97.4 | NA | NA | NA | NA |
| HJ1D73 | NA | NA | NA | 60.8 | 87.7 | 98.9 | 82.5 | 36.2 | 90.0 | 77.9 | 68.0 | 76.7 |
| HMJ1D1 | NA | NA | NA | 83.2 | 95.4 | 95.6 | 91.4 | 74.1 | 81.2 | 76.8 | 77.4 | 85.8 |
| HMJ1D2 | NA | NA | NA | NA | NA | 82.0 | 82.0 | NA | NA | NA | NA | NA |
| MJ1D11 | NA | NA | NA | NA | 86.9 | 82.0 | 84.4 | NA | 79.2 | 79.2 | 79.2 | 82.3 |

TABLE 4
Synthesis of the experiments carried out to test the siRNAs directed against human JAK3.

| | Fluorescence | | | | | | | | |
|---|---|---|---|---|---|---|---|---|---|
| | Quantification (Cellinsight | | | | | | | | |
| | Microscope) | | | | | | | | |
| | | | Weighted | | | | | | |
| | | | Average | Western Blot | | | | | |
| | | PC3* | Zscore | | | PC3* | PC3* | PC3* | Average |
| | PC3* | with | Exp w/ | PC3* | PC3* | with | with | with | Inhibition |
| | with | 1 nM | 10 nM = 1 | with | with | 0.2 nM | 0.2 nM | 0.2 nM | of JAK3 |
| | 10 nM | siRNA | and Exp | 1 nM | 0.2 nM | siRNA | siRNA | siRNA | at 0.2 nM |
| | siRNA | Z- | w/1 nM = 2 | siRNA | siRNA | Exp1 | Exp2 | Exp3 | Exp1-2-3 |
| | siRNA | | | | | | | | |
| | | | | | | | | | Avg. |
| | | | Weighted | % | % | % | % | % | Inhib. |
| | | | Average | inhib. | inhib. | inhib. | inhib. | inhib. | Jak3 at |
| | Zscore | Zscore | Zscore | Jak3 | Jak3 | Jak3 | Jak3 | Jak3 | 0.2 nM |
| AS | 0.0 | 0.0 | 0.0 | 0.0 | 0.0 | 0.0 | 0.0 | 0.0 | 0.0 |
| HJ3A52 | −0.5 | −1.2 | −1.0 | 61.0 | 72.0 | NA | NA | NA | NA |
| HJ3A53 | −1.0 | −1.2 | −1.1 | 78.9 | 85.8 | 85.4 | 74.3 | 85.8 | 81.8 |
| HJ3A54 | −0.9 | −1.3 | −1.2 | 58.5 | 74.2 | NA | NA | NA | NA |
| HJ3Q1 | −1.0 | −2.2 | −1.8 | 78.7 | 83.3 | 83.0 | 82.3 | 77.7 | 81.0 |
| HJ3Q2 | 1.2 | 1.0 | 1.0 | −30.2 | −0.4 | NA | NA | NA | NA |
| HJ3Q6 | −0.4 | −0.8 | −0.7 | 59.8 | 52.1 | 38.3 | 3.0 | NA | 20.6 |
| HJ3Q11 | 1.0 | 1.2 | 1.1 | 20.1 | −4.3 | NA | NA | NA | NA |
| HJ3H9 | −0.5 | −1.2 | −1.0 | 83.0 | 72.4 | 81.6 | 83.3 | 89.7 | 84.9 |
| HJ3H10 | −0.1 | −1.1 | −0.8 | 80.7 | 68.2 | 40.8 | 76.7 | 74.8 | 64.1 |
| HJ3H11 | −0.3 | −0.7 | −0.5 | 74.0 | 73.3 | NA | NA | NA | NA |
| HJ3H12 | −0.4 | −1.1 | −0.8 | 83.3 | 82.6 | 79.8 | 23.2 | 93.3 | 65.4 |
| HJ3D1 | −0.5 | −1.8 | −1.3 | 67.9 | 70.6 | 77.7 | 75.1 | 85.0 | 79.3 |
| HJ3D5 | −0.5 | −3.3 | −2.4 | 53.9 | 64.1 | 81.3 | 78.3 | 86.7 | 82.1 |
| HJ3D8 | −0.3 | 0.6 | 0.3 | 23.2 | 57.8 | 65.0 | 58.1 | 71.1 | 64.7 |
| HJ3D11 | −0.5 | −1.7 | −1.3 | 31.2 | 62.3 | 74.2 | 72.2 | 82.1 | 76.2 |
| HJ3D12 | −1.2 | −2.2 | −1.9 | 57.7 | 63.3 | 76.5 | 81.1 | 73.2 | 76.9 |
| HJ3D15 | −0.3 | −1.2 | −0.9 | 54.7 | 75.0 | 74.1 | 75.8 | 79.6 | 76.5 |
| HJ3D18 | −0.3 | −1.2 | −0.9 | 20.8 | 63.8 | 40.3 | 34.8 | 59.0 | 44.7 |
| HJ3D23 | −0.4 | −2.2 | −1.6 | 44.3 | 65.2 | 80.1 | 81.7 | 80.9 | 80.9 |
| HJ3D38 | 0.0 | −1.8 | −1.2 | 56.7 | 69.4 | 81.6 | 64.7 | 78.4 | 74.9 |
| HJ3D41 | −0.4 | −1.8 | −1.3 | 71.3 | 83.1 | 84.2 | 88.3 | 87.3 | 86.6 |
| HMJ3D1 | −0.1 | −1.8 | −1.2 | 76.1 | 68.6 | 85.5 | 88.3 | 77.7 | 83.8 |
| | | | | | | | | | Weighted |
| | | | | | | | | | Efficacy |
| | | | | | | | | | Score with |
| | | | | | | | | | HCS@1 |
| | | | | | | | | | FACS@1, |
| | FACS | | | | RT-QPCR | | | | WB_E3-1 |
| | PC3* | PC3* | PC3* | | PC3* | PC3* | PC3* | Average | 0.2 nM@2, |
| | with | with | with | Average | with | with | with | Inhibition | WB_E8 |
| | 0.2 nM | 0.2 nM | 0.2 nM | inhibition | 0.2 nM | 0.2 nM | 0.2 nM | of JAK3 | 0.2 nM@3, |
| | siRNA | siRNA | siRNA | at 0.2 nM | siRNA | siRNA | siRNA | at 0.2 nM | QPCR |
| | Exp1 | Exp2 | Exp3 | Exp1-2-3 | Exp1 | Exp2 | Exp3 | Exp1-2-3 | 0.2@3 |
| | siRNA | | | | | | | | |
| | | | | Avg. | | | | Avg. | |
| | % | % | % | Inhib. | inhib. | inhib. | inhib. | Inhib. | Weighted |
| | inhib. | inhib. | inhib | Jak3 at | Jak3 | Jak3 | Jak3 | Jak3 at | Efficacy |
| | Fluo. | Fluo. | Fluo. | 0.2 nM | mRNA | mRNA | mRNA | 0.2 nM | Score |
| AS | 0.0 | 0.0 | 0.0 | 0.0 | 0.0 | 0.0 | 0.0 | 0.0 | 0.0 |
| HJ3A52 | NA | NA | NA | NA | NA | NA | NA | NA | NA |
| HJ3A53 | 54.1 | 51.3 | 55.0 | 53.5 | 94.1 | 89.6 | 93.1 | 92.3 | 74.8 |
| HJ3A54 | NA | NA | NA | NA | NA | NA | NA | NA | NA |
| HJ3Q1 | 54.1 | 57.1 | 64.4 | 58.5 | 89.2 | 60.3 | 93.6 | 81.0 | 71.3 |
| HJ3Q2 | NA | NA | NA | NA | NA | NA | NA | NA | NA |
| HJ3Q6 | NA | NA | NA | NA | NA | NA | NA | NA | NA |
| HJ3Q11 | NA | NA | NA | NA | NA | NA | NA | NA | NA |
| HJ3H9 | NA | NA | NA | NA | NA | NA | NA | NA | NA |
| HJ3H10 | NA | NA | NA | NA | NA | NA | NA | NA | NA |
| HJ3H11 | NA | NA | NA | NA | NA | NA | NA | NA | NA |
| HJ3H12 | 51.4 | 52.4 | 56.4 | 53.4 | 86.7 | 95.3 | 95.2 | 92.4 | 69.3 |
| HJ3D1 | 49.2 | 50.8 | 54.0 | 51.3 | 96.8 | 81.7 | 93.5 | 90.7 | 70.4 |
| HJ3D5 | 42.1 | 45.0 | 51.5 | 46.2 | 92.4 | 67.2 | 95.2 | 84.9 | 67.8 |
| HJ3D8 | NA | NA | NA | NA | NA | NA | NA | NA | NA |
| HJ3D11 | NA | NA | NA | NA | 92.0 | 93.6 | 93.5 | 93.0 | NA |
| HJ3D12 | 44.8 | 43.9 | 52.0 | 46.9 | 77.6 | 86.6 | 87.9 | 84.0 | 65.8 |
| HJ3D15 | NA | NA | NA | NA | 78.3 | 79.4 | 87.0 | 81.6 | NA |
| HJ3D18 | NA | NA | NA | NA | NA | NA | NA | NA | NA |
| HJ3D23 | 41.0 | 37.0 | 45.0 | 41.0 | 90.4 | 97.1 | 94.4 | 94.0 | 69.8 |
| HJ3D38 | 39.9 | 41.3 | 48.5 | 43.2 | 86.0 | 40.8 | 91.9 | 72.9 | 62.7 |
| HJ3D41 | 47.5 | 51.3 | 53.5 | 50.8 | 89.7 | 69.2 | 89.1 | 82.7 | 72.6 |
| HMJ3D1 | 48.6 | 55.6 | 58.4 | 54.2 | 92.1 | 91.1 | 91.1 | 91.4 | 71.8 |

*PC3 = PC3-HsJak3-GFP cells E8 clone

### Example 2: Efficacy of the siRNAs

a. Analysis to Ascertain the Specificity of Gene Inhibition:

The specificity of the siRNA sequences was evaluated using various molecular biology approaches, by analysing via RTqPCR the gene expression of the 4 members of the JAK protein family: JAK1, JAK2, JAK3 and TYK2. The experiments were performed with a concentration of 10 nM siRNA in order to promote potential non-specific effects which are known to be dose-dependent.

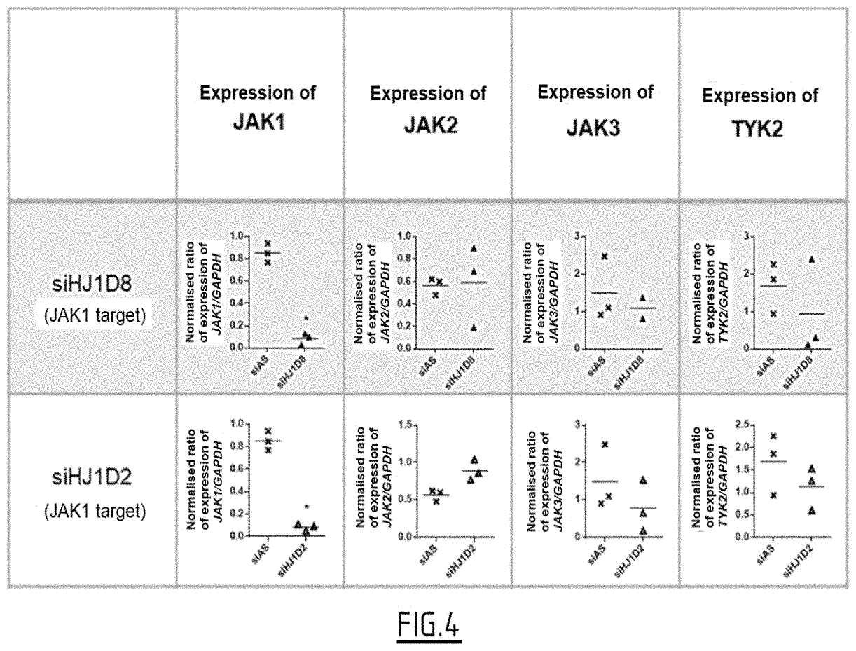

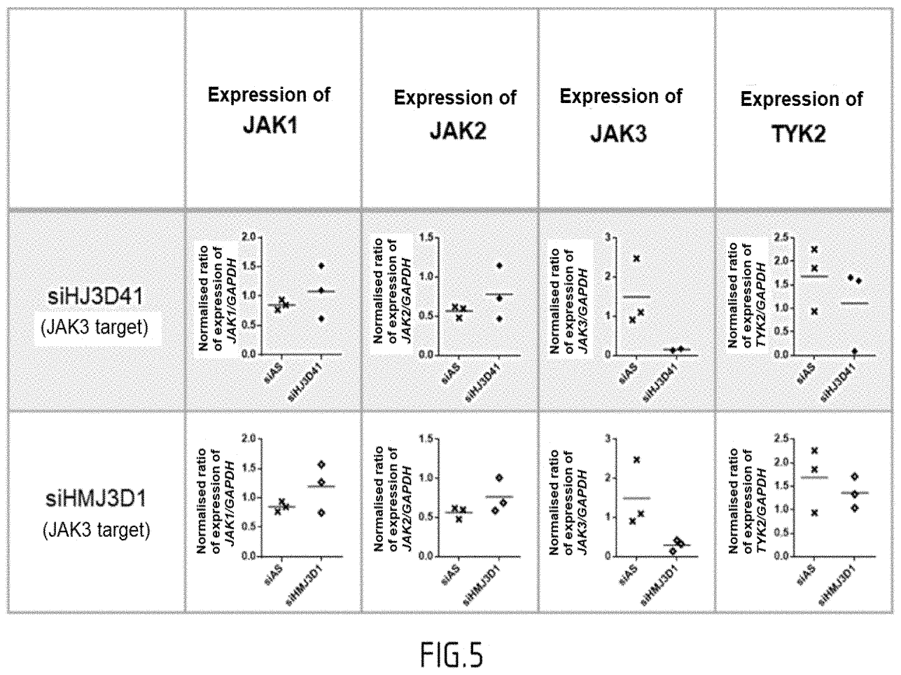

The Caco-2 cells were transfected with 10 nM of siRNA targeting JAK1 or JAK3 using Lipofectamine RNAimax for 48 hours. The siRNAs siHJ1D8 and siHJ1D2 target JAK1 and the siRNAs siHJ3D41 and HMJ3D1 target JAK3. The RNAs were extracted, reverse transcribed into cDNA and tested by Quantitative Reverse Transcription Polymerase Chain Reaction (RTqPCR) technique. The gene Glyceraldehyde-3-phosphate dehydrogenase (GAPDH) was used as an internal gene, and the cells not subjected to any treatment served as an external calibrator. The siRNA “siAS” is a transfection control that has no homology to the human genome. The results of 3 independent experiments for measuring the level of JAK1, JAK2, JAK3 or TYK2 gene expression in Caco-2 cells are shown in FIGS. 4 and 5.

These results were also confirmed at the protein level by Western blot testing. The PC3 cells transfected with a vector pCDNA3.1 in which the sequence encoding JAK3 fused at its C-terminus with the GFP, were transfected with 10 nM of siRNA targeting JAK1 or JAK3 using Lipofectamine RNAimax for 48 hours in order to enable monitoring of the JAK3 protein in particular. The JAK-siRNAs only impact the expression of their target.

b. Analysis to Ascertain the Sensitivity:

Investigation was performed to determine the minimum effective dose of siRNA necessary to block the endogenous expression of the JAK1 or JAK3 genes (si-JAK1: <12.5 pM in the human epithelial line PC3; si-JAK3: <1 nM in the human epithelial line PC3 overexpressing JAK3 by means of the introduction of a plasmid).

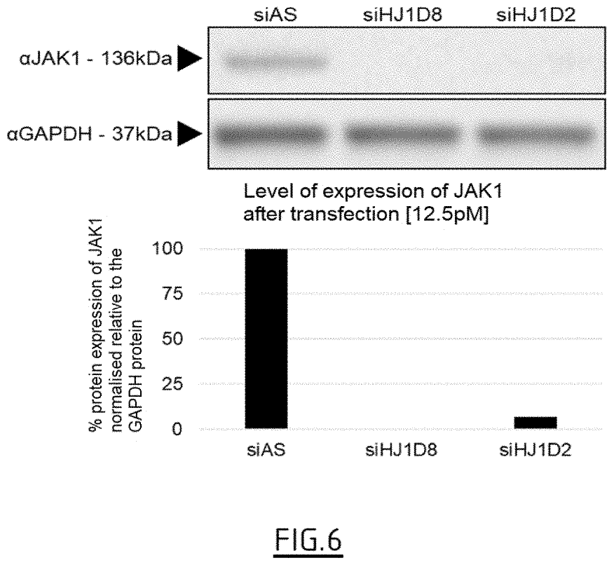

The PC3 cells were transfected with 12.5 pM of siRNA using Lipofectamine RNAimax for 48 hours. The cells were lysed using a standard conventional lysis buffer. By means of Western blotting, the protein lysates were then analysed for the expression of JAK1 and Glyceraldehyde-3-phosphate dehydrogenase (GAPDH). FIG. 6 represents the quantification of the expression of JAK1, normalised to GAPDH.

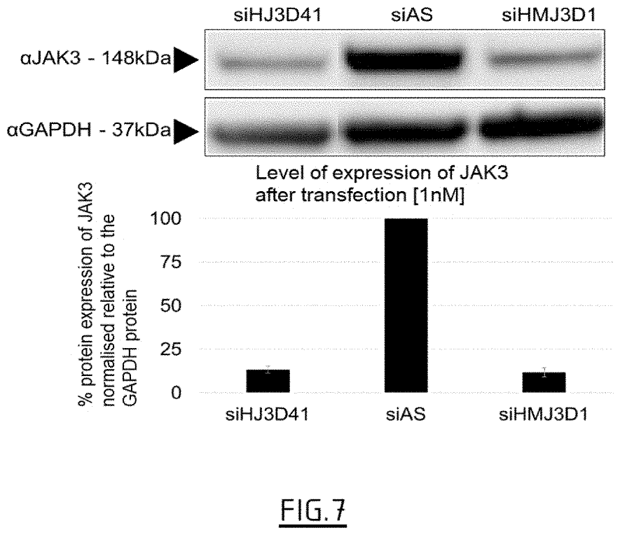

The PC3 cells overexpressing JAK3-GFP were transfected with 1 nM of siRNA using Lipofectamine RNAimax for 48 hours. The cells were lysed using a standard conventional lysis buffer. By means of Western blotting, the protein lysates were then analysed for the expression of JAK3 and GAPDH. FIG. 7 shows the quantification of the expression of JAK3.

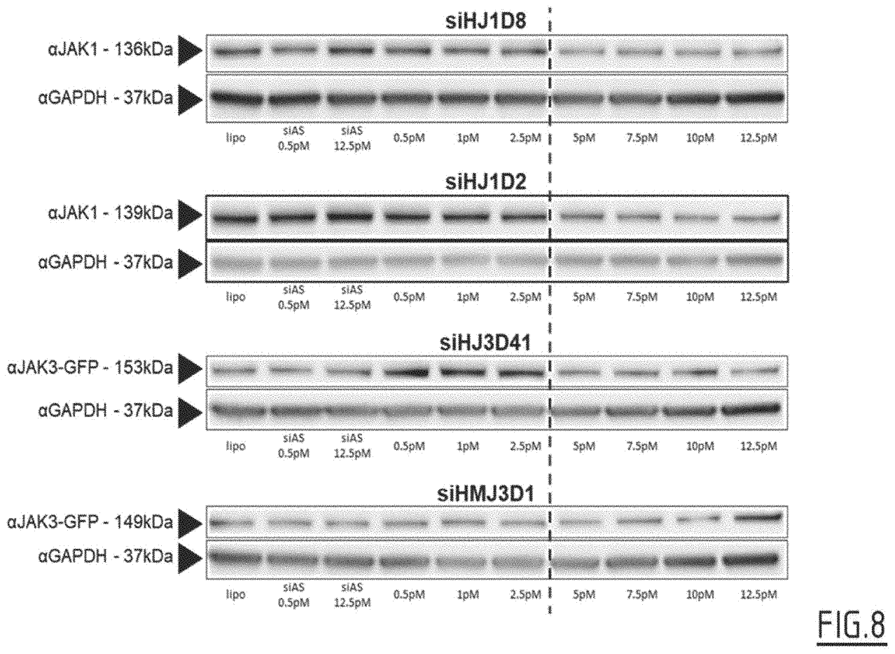

The PC3 cells overexpressing JAK3-GFP were finally transfected with different concentrations of siRNA (from 0.5 pM to 12.5 pM) using Lipofectamine RNAimax for 48 hours. The cells were lysed using a standard conventional lysis buffer. By means of Western blotting, the protein lysates were then analysed for the expression of JAK3 or JAK1 and GAPDH. FIG. 8 presents the results of a Western blot experiment representative of 3 independent experiments.

The siRNA sequences provided therefore remain effective at doses that are well below those recommended for the sequences already being commercially marketed (rarely less than 10 nM). At a concentration of 5 pM, the sequences generated indeed continue to exhibit high JAK inhibition efficacy for inhibiting the expression of JAK1 or JAK3 (see FIG. 8).

c. Investigation of Undesirable/Adverse Effects:
i. microRNA Effects

The inventors investigated the presence of 3′ UTR nucleotide sequences from the entire human genome that can be recognised by the “seed” sequences of siRNAs.

TABLE 5
The number of times where the “seed” sequence of si-JAK1 could exhibit
sequence complementarity with a 3′UTR nucleotide sequence was
investigated in silico over the entire human genome.

| | Predicted Sequence Complementarity | | |
|---|---|---|---|
| siRNA | 1 hit | 2 hits | >=3 hits |
| HJ1D2 | 339 | 7 | 0 |
| HJ1D8 | 287 | 13 | 0 |

TABLE 6
The number of times where the “seed”
sequence of si-JAK3 could exhibit
sequence complementarity with a 3′UTR nucleotide
sequence was investigated in silico over the entire human genome.

| | Predicted Sequence Complementarity | | |
|---|---|---|---|
| siRNA | 1 hit | 2 hits | >=3 hits |
| HJ3D41 | 527 | 8 | 1 |
| HMJ3D1 | 1161 | 48 | 3 |

The results obtained have been synthesised in Table 5 for the si-JAK1s and in Table 6 for the si-JAK3s. The selected si-JAKs present a very moderate risk of activating a microRNA response.

ii. Potential Direct Effects on Other Genes

The si-JAK sequence homology for all of the human genes was analysed in silico by making use of the Basic Local Alignment Search Tool/Nucleotide, ie BLASTN 2.7.0+ program (freely available) from the US National Center for Biotechnology Information (NCBI). This approach made it possible to study the sequence homologies of the passenger and guide strands of the siRNAs with the entire human genome.

Potential targets having partial sequence homology with the siRNAs were thus identified.

The set of all genes having partial sequence homology to siHJ1D2 has been compiled in Table 7.

TABLE 7
The genes exhibiting partial sequence homology
with the passenger and guide strands of
siHJ1D2 have been compiled in this table.
The “seed” sequence of siHJ1D2 is
underlined, the sequence homology between
the gene and siHJ1D2 is in bold italics.

| | | | Position of |
|---|---|---|---|
| | | | Sequence |
| | | | Homology |
| | | | (“Seed” |
| | | | Sequence of |
| | | | siHJ1D2 |
| Gene | % | Homologous | (SEQ ID NO: 6) |
| Name | Homology | Nucleotides | Underlined) |
| NPBWR2 | 80% | 17/17 | UU [symbol] [symbol] |
| | | | [symbol] |
| FECH | 71% | 15/15 | UUCG [symbol] [symbol] |
| | | | [symbol] |
| TAF4 | 71% | 15/15 | U [symbol] [symbol] |
| | | | UCCAU |
| ADAMTSL2 | 66% | 14/14 | [symbol] [symbol] |
| | | | AAUCCAU |
| ABLIM 1 | 66% | 14/14 | UUCGUC [symbol] [symbol] |
| | | | [symbol] U |

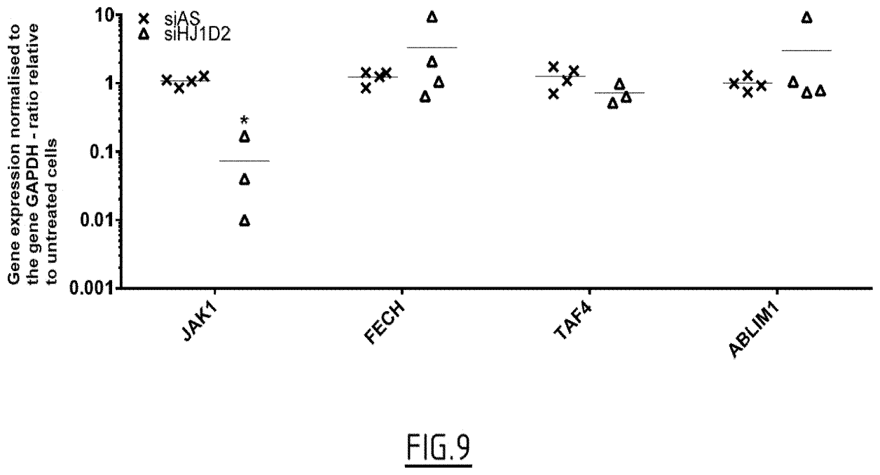

Certain potential targets found by the in silico analysis have been experimentally validated by RTqPCR. In order to do this, the T47D cells were transfected with 10 nM of siRNA using Lipofectamine RNAimax for 48 hours. The RNAs were extracted, reverse transcribed into cDNA and tested by Quantitative Reverse Transcription Polymerase Chain Reaction (RTqPCR) technique. The gene Glyceraldehyde-3-phosphate dehydrogenase (GAPDH) was used as an internal gene, and the cells not subjected to any treatment served as an external calibrator. The siRNA “siAS” is a transfection control that has no homology to the human genome. The results shown in FIG. 9 represent 4 independent dot plot experiments with the mean overprinted.

Although partial sequence homology between the genes FECH, TAF4 and ABLIM1 and the siHJ1D2 has been detected in silico, this latter siRNA has no impact on the expression of these genes.

The set of all genes having partial sequence homology to siHJ1D8 has been compiled in Table 8.

TABLE 8
The genes exhibiting partial sequence
homology with the passenger and
guide strands of siHJ1D8 have been
compiled in this table. The “seed”
sequence of siHJ1D8 is underlined,
the sequence homology between the
gene and siHJ1D8 is in bold
italics.

| | | | Position of Sequence |
|---|---|---|---|
| | | Homol- | Homology (“Seed” |
| | % | ogous | Sequence of siHJ1D8 |
| Gene | Homol- | Nucleo- | (SEQ ID NO: 8) |
| Name | ogy | tides | Underlined) |
| ZFYVE1 | 71% | 15/15 | UCGC [symbol] [symbol] [symbol] UU |
| ZNF782 | 66% | 14/14 | UCGC [symbol] [symbol] [symbol] CUU |
| TPMT | 66% | 14/14 | UCGCUUG [symbol] [symbol] |
| OAS1 | 66% | 14/14 | UCGCUUG [symbol] [symbol] |
| FLO11- | 61% | 13/13 | UCGCU [symbol] [symbol] [symbol] CUU |
| like | | | |
| MFSD14B | 61% | 13/13 | UCGCU [symbol] [symbol] [symbol] CUU |
| BACH2 | 61% | 13/13 | UCG [symbol] [symbol] UCCUU |
| CADPS2 | 61% | 13/13 | UCGCU [symbol] [symbol] [symbol] CUU |
| ARL14EP | 61% | 13/13 | [symbol] [symbol] AUGUCCUU |
| NCSTN | 61% | 13/13 | UCGCUUGU [symbol] [symbol] |
| RNH1 | 61% | 13/13 | UCGCUUGU [symbol] [symbol] |
| MEGF10 | 61% | 13/13 | UCGCUU [symbol] [symbol] U |
| FAM160A1 | 76% | 13/13 | UCGCUU [symbol] [symbol] UU |
| ZDHHC23 | 61% | 13/13 | UCGC [symbol] [symbol] [symbol] CCUU |

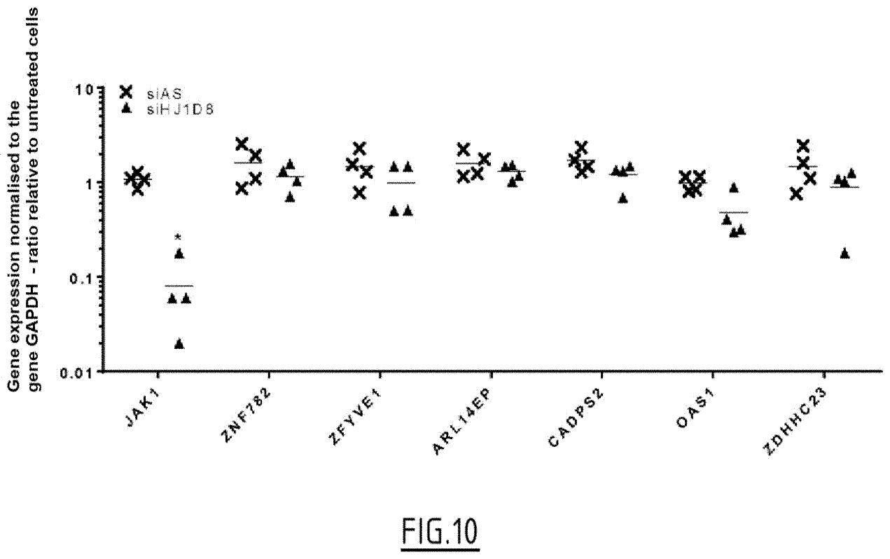

Certain potential targets found by the in silico analysis have been experimentally validated by RTqPCR. In order to do this, the T47D cells were transfected with 10 nM of siRNA using Lipofectamine RNAimax for 48 hours. The RNAs were extracted, reverse transcribed into cDNA and tested by Quantitative Reverse Transcription Polymerase Chain Reaction (RTqPCR) technique. The gene Glyceraldehyde-3-phosphate dehydrogenase (GAPDH) was used as an internal gene, and the cells not subjected to any treatment served as an external calibrator. The siRNA “siAS” is a transfection control that has no homology to the human genome. The results shown in FIG. 10 represent 4 independent dot plot experiments with the mean overprinted.

Although partial sequence homology between the genes ZNF782, ZFYVE1, ARL14EP, CADPS2, OAS1 and ZDHHC23, and the siHJ1D8 has been detected in silico, this latter siRNA has no impact on the expression of these genes.

The set of all genes having partial sequence homology to siHJ3D41 has been compiled in Table 9.

TABLE 9
The genes exhibiting partial sequence homology
with the passenger and guide strands of
siHJ3D41 have been compiled in this table.
The “seed” sequence of siHJ3D41 is
underlined, the sequence homology between
the gene and siHJ3D41 is in bold italics.

| | | | Position of Sequence |
|---|---|---|---|
| | | Homol- | Homology (“Seed” |
| | % | ogous | Sequence of siHJ3D41 |
| Gene | Homol- | Nucleo- | (SEQ ID NO: 2) |
| Name | ogy | tides | Underlined) |
| ZSWIM4 | 76% | 16/16 | UA [symbol] [symbol] [symbol] GCA |
| LRCH2 | 71% | 15/15 | U [symbol] [symbol] UCGCA |
| RNPEP | 66% | 14/14 | UAC [symbol] [symbol] [symbol] CGCA |
| PHF21A | 66% | 14/14 | UAC [symbol] [symbol] [symbol] CGCA |

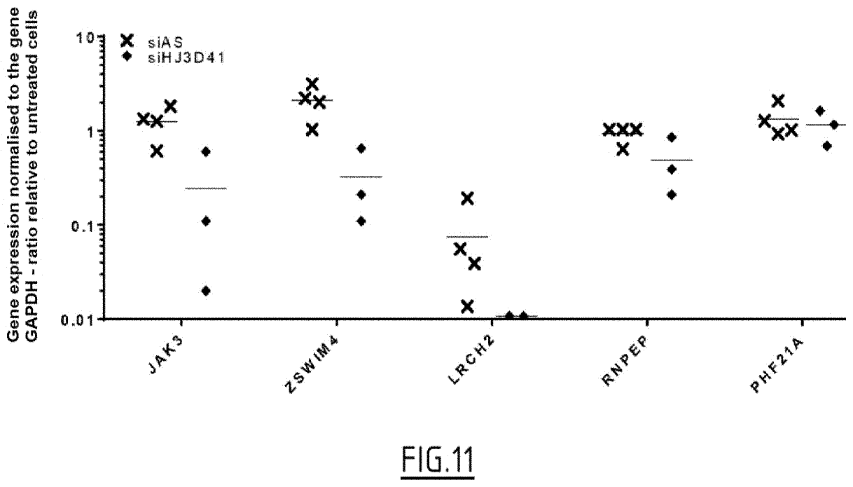

Certain potential targets found by the in silico analysis have been experimentally validated by RTqPCR. In order to do this, the T47D cells (genes ZSWIM4, RNPEP and PHF21A) or PC3 cells (the gene LRCH2, because it is not expressed by the T47D cells and therefore could not be studied in this line) were transfected with 10 nM of siRNA using Lipofectamine RNAimax for 48 hours. The RNAs were extracted, reverse transcribed into cDNA and tested by Quantitative Reverse Transcription Polymerase Chain Reaction (RTqPCR) technique. The gene Glyceraldehyde-3-phosphate dehydrogenase (GAPDH) was used as an internal gene, and the cells not subjected to any treatment served as an external calibrator. The siRNA “siAS” is a transfection control that has no homology to the human genome. The results shown in FIG. 11 represent 4 independent dot plot experiments with the mean overprinted.

The silHJ3D41 appears to decrease the gene expression of ZSWIM4 and LRCH2, but not that of RNPEP and PHF21A.

The set of all genes having partial sequence homology to siHMJ3D1 has been compiled in Table 10.

TABLE 10
The genes exhibiting partial sequence homology
with the passenger and guide strands of
siHMJ3D41 have been compiled in this table.
The “seed” sequence of siHMJ3D41 is underlined,
the sequence homology between the gene and
siHMJ3D41 is in bold italics.

| | | | Position of Sequence |
|---|---|---|---|
| | | Homol- | Homology (“Seed” |
| | % | ogous | Sequence of siHMJ3D1 |
| Gene | Homol- | Nucleo- | (SEQ ID NO: 4) |
| Name | ogy | tides | Underlined) |
| KANK3 | 71% | 15/15 | UAG [symbol] [symbol] [symbol] CUG |
| DNAH9 | 66% | 14/14 | UAG [symbol] [symbol] [symbol] GCUG |
| FARP1 | 66% | 14/14 | UAGC [symbol] [symbol] [symbol] CUG |
| TMEM120B | 85% | 17/18 | UAGC [symbol] [symbol] [symbol] |
| FAM213A | 66% | 14/14 | UAG [symbol] [symbol] [symbol] GCUG |
| CHD7 | 61% | 13/13 | UAGC [symbol] [symbol] GCUG |
| TMEM267 | 61% | 13/13 | UA [symbol] [symbol] ACGCUG |
| MAST4 | 61% | 13/13 | UAG [symbol] [symbol] CGCUG |
| DGKQ | 61% | 13/13 | UAG [symbol] [symbol] CGCUG |
| GSDMD | 61% | 13/13 | UAGCGGCA [symbol] [symbol] |
| POLDIP3 | 61% | 13/13 | U [symbol] [symbol] CACGCUG |
| GRIP2 | 61% | 13/13 | UAGCGGCA [symbol] [symbol] |
| TEKT4 | 61% | 13/13 | UAG [symbol] [symbol] CGCUG |
| HAUS8 | 61% | 13/13 | UAGC [symbol] [symbol] [symbol] CUG |
| YJEFN3 | 61% | 13/13 | UAGCG [symbol] [symbol] [symbol] CUG |
| FCH01 | 61% | 13/13 | U [symbol] [symbol] CACGCUG |
| ELAC2 | 61% | 13/13 | UAG [symbol] [symbol] CGCUG |
| SGSM3 | 61% | 13/13 | UAGCGGCA [symbol] [symbol] |

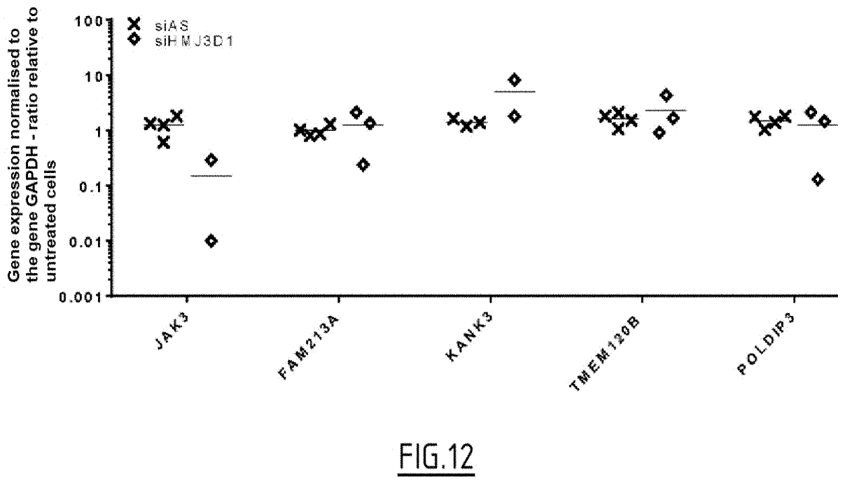

Certain potential targets found by the in silico analysis have been experimentally validated by RTqPCR. In order to do this, the T47D cells were transfected with 10 nM of siRNA using Lipofectamine RNAimax for 48 hours. The RNAs were extracted, reverse transcribed into cDNA and tested by Quantitative Reverse Transcription Polymerase Chain Reaction (RTqPCR) technique. The gene Glyceraldehyde-3-phosphate dehydrogenase (GAPDH) was used as an internal gene, and the cells not subjected to any treatment served as an external calibrator. The siRNA “siAS” is a transfection control that has no homology to the human genome. The results shown in FIG. 12 represent 4 independent dot plot experiments with the mean overprinted.

Although partial sequence homology between the genes FAM213A, KANK3, TMEM120B and POLDIP3, and the siHMJ3D1 has been detected in silico, this latter siRNA has no impact on the expression of these genes.

iii. Phenotypic Effects

The impact of the use of siRNAs on major cellular functions (proliferation, apoptosis, and ATP metabolism) was analysed.

The Caco-2 cells were transfected with 10 nM of siRNA using Lipofectamine RNAimax.

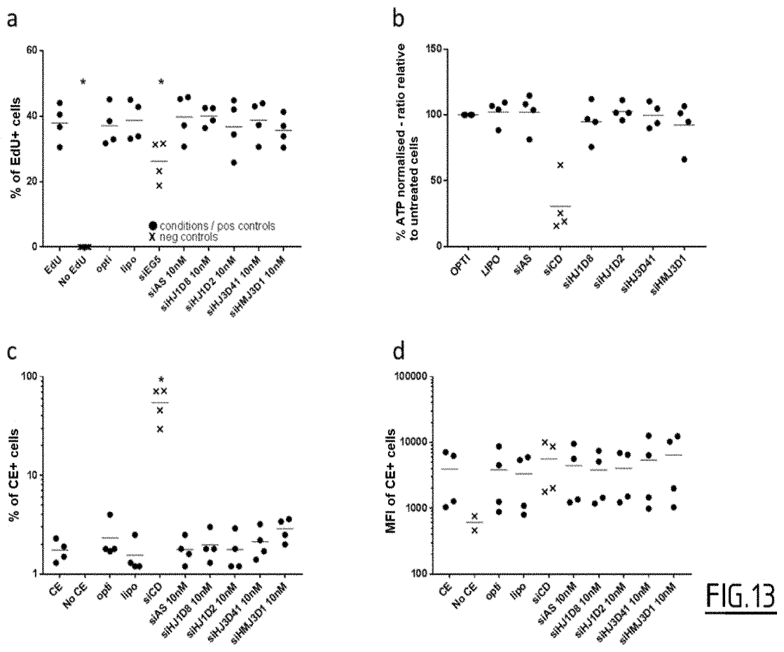

The transfected cells were then incubated for a period of 5 hours in the presence of EdU (5 μM), which is a fluorescent agent that brings about intercalation in the DNA of cells that proliferate. The cells were thereafter detached and then permeabilised before being analysed by flow cytometry. The siRNA targeting the EG5 gene is an experimental internal control since this gene is directly involved in cell proliferation. The results in FIG. 13a represent 4 independent dot plot experiments with the mean overprinted; “*” signifies a significant p-value in a paired non-parametric statistical test. The siRNAs of interest have no impact on proliferation.

Furthermore, after 48 hours of transfection, the level of cellular ATP was measured using a Vialight kit (FIG. 13b.)

During the transfection, the cells were placed in the presence of CellEvent, a marker of activated capase-3 and capase-7. This marker makes it possible to quantify apoptosis. The cells were analysed after permeabilisation by flow cytometry. FIG. 13c represents the percentage of cells in apoptosis while FIG. 13d represents the number of fluorescent molecules on the surface of positive cells.

The siRNAs did not reduce cell proliferation, or increase apoptosis, or alter ATP metabolism in any statistically significant manner.

1. (canceled)

2. A method of preventing and/or treating a disease associated with a Janus kinase (JAK) gain of function disruption of the Janus kinase 1 (JAK1) or Janus kinase 3 (JAK3) signalling pathway, the method comprising:
administering to a subject in need thereof a therapeutically effective amount of at least one the double-stranded (ds) ribonucleic acid (RNA) of claim 7.

3. The method of claim 2, wherein the disease comprises an immune-mediated inflammatory disease, cancer, or a combination of two or more of any of these.

4. The method of claim 2, wherein the dsRNA reduces the expression of Janus kinase 3 (JAK3) and wherein:
the sense strand comprises the nucleotide sequence SEQ ID NO: 1 and the antisense strand comprises the nucleotide sequence SEQ ID NO: 2; or
the sense strand comprises the nucleotide sequence SEQ ID NO: 3 and the anti sense strand comprises the nucleotide sequence SEQ ID NO: 4.

5. The method of claim 2, wherein the dsRNA reduces the expression of Janus kinase 1 (JAK1) and wherein:
the sense strand comprises the nucleotide sequence SEQ ID NO: 5 and the antisense strand comprises the nucleotide sequence SEQ ID NO: 6; or
the sense strand comprises the nucleotide sequence SEQ ID NO: 7 and the antisense strand comprises the nucleotide sequence SEQ ID NO: 8.

6. The method of claim 2, wherein the subject is a human subject.

7. A double-stranded (ds) ribonucleic acid (RNA) comprising a sense strand and an antisense strand, adapted for use in human therapy, wherein:
the sense strand comprises the nucleotide sequence SEQ ID NO: 1 and the antisense strand comprises the nucleotide sequence SEQ ID NO: 2; or
the sense strand comprises the nucleotide sequence SEQ ID NO: 3 and the antisense strand comprises the nucleotide sequence SEQ ID NO: 4; or
the sense strand comprises the nucleotide sequence SEQ ID NO: 5 and the antisense strand comprises the nucleotide sequence SEQ ID NO: 6; or
the sense strand comprises the nucleotide sequence SEQ ID NO: 7 and the antisense strand comprises the nucleotide sequence SEQ ID NO: 8.

8. A pharmaceutical composition comprising:
a pharmaceutically acceptable carrier; and
a double-stranded (ds) ribonucleic acid (RNA) comprising a sense strand and an antisense strand wherein:
the sense strand comprises the nucleotide sequence SEQ ID NO: 1 and the antisense strand comprises the nucleotide sequence SEQ ID NO: 2; or
the sense strand comprises the nucleotide sequence SEQ ID NO: 3 and the antisense strand comprises the nucleotide sequence SEQ ID NO: 4; or
the sense strand comprises the nucleotide sequence SEQ ID NO: 5 and the antisense strand comprises the nucleotide sequence SEQ ID NO: 6; or
the sense strand comprises the nucleotide sequence SEQ ID NO: 7 and the antisense strand comprises the nucleotide sequence SEQ ID NO: 8.

9. An in vitro method for inhibiting the expression of Janus kinase 1 (JAK1) or Janus kinase 3 (JAK3) in a cell, the method comprising:
(a) introducing into the cell a double-stranded (ds) ribonucleic acid (RNA), said dsRNA comprising a sense strand and an antisense strand wherein:
the sense strand comprises the nucleotide sequence SEQ ID NO: 1 and the antisense strand comprises the nucleotide sequence SEQ ID NO: 2; or
the sense strand comprises the nucleotide sequence SEQ ID NO: 3 and the antisense strand comprises the nucleotide sequence SEQ ID NO: 4; or
the sense strand comprises the nucleotide sequence SEQ ID NO: 5 and the antisense strand comprises the nucleotide sequence SEQ ID NO: 6; or
the sense strand comprises the nucleotide sequence SEQ ID NO: 7 and the antisense strand comprises the nucleotide sequence SEQ ID NO: 8; and
(b) maintaining the cell produced in the introducing (a) for a period of time sufficient to achieve degradation of the mRNA of a JAK1 or JAK3 gene, thereby inhibiting the expression of JAK1 or JAK3 in the cell.

10. The method of claim 9, wherein, in the introducing (a), the dsRNA is brought into contact with the cell at a concentration in a range of from 5 pM to 10 nM.

11. The method of claim 2, wherein the nucleotide at the 5′ end of the antisense strand of dsRNA is phosphorylated.

12. The method of claim 2, wherein each strand of the dsRNA comprises at most 24 nucleotides.

13. The method of claim 2, wherein the two strands of the dsRNA are of identical length.

14. The method of claim 2, wherein the dsRNA comprises at most 60 nucleotides.

15. The dsRNA of claim 7, wherein the nucleotide at the 5′ end of the antisense strand of dsRNA is phosphorylated.

16. The dsRNA of claim 7, wherein each strand of the dsRNA comprises at most 24 nucleotides.

17. The dsRNA of claim 7, wherein the two strands of the dsRNA are of identical length.

18. The dsRNA of claim 7, wherein the dsRNA comprises at most 60 nucleotides.

19. The composition of claim 8, wherein the nucleotide at the 5′ end of the antisense strand of dsRNA is phosphorylated.

20. The composition of claim 8, wherein each strand of the dsRNA comprises at most 24 nucleotides.

21. The composition of claim 8, wherein the two strands of the dsRNA are of identical length.


SEQUENCE LISTING (8 sequences)

SEQ ID NO: 1 - RNA, 21 nt, Artificial Sequence
  brin sens siARN HJD41
cgacuuuccagaaaucguaga

SEQ ID NO: 2 - RNA, 21 nt, Artificial Sequence
  brin antisens siARN HJD41
uacgauuucuggaaagucgca

SEQ ID NO: 3 - RNA, 21 nt, Artificial Sequence
  brin sens siARN HMJ3D1
gcguggagcugugccgcuaug

SEQ ID NO: 4 - RNA, 21 nt, Artificial Sequence
  brin antisens siARN HMJ3D1
uagcggcacagcuccacgcug

SEQ ID NO: 5 - RNA, 21 nt, Artificial Sequence
  brin sens siARN HJ1D2
ggauuacaaggaugacgaagg

SEQ ID NO: 6 - RNA, 21 nt, Artificial Sequence
  brin antisens siARN HJ1D2
uucgucauccuuguaauccau

SEQ ID NO: 7 - RNA, 21 nt, Artificial Sequence
  brin sens siARN HJ1D8
ggacaucagcuacaagcgaua

SEQ ID NO: 8 - RNA, 21 nt, Artificial Sequence
  brin antisens siARN HJ1D8
ucgcuuguagcugauguccuu